(19) United States
(12) Patent Application Publication
(10) Pub. No.: US 2004/0064025 A1
(43) Pub. Date: Apr. 1, 2004

(54) Medical electrode system and method of use

(76) Inventor: Peter James Taylor, Victoria (AU)

Correspondence Address: Edwin D Schindler, Five Hirsch Avenue, P O Box 966, Coram, NY, 11727-0966, US

(21) Appl. No.: 10/398,589

(22) PCT Filed: Oct. 3, 2001
(86) PCT No.: PCT/AU01/01240

(30) Foreign Application Priority Data
Oct. 3, 2000 (AU) PR0538

(51) Int. Cl. A61B 5/04

(52) U.S. Cl. 600/382

(57) ABSTRACT

An electrode assembly (10) for connection to a patient's body comprising electrodes (20) and a transmitter (50) for operable connection to the electrode assembly (10) to receive the output therefrom and transmit these to a receiver associated with monitoring equipment.

This invention relates to a medical electrode system and method of use.

It is quite conventional for patients in medical situations to be provided with a number of electrodes which are connected to measuring equipment often by cables associated with the equipment which are brought into connection with the electrodes after the electrode is connected on the patients body.

For some equipment the patient may carry a substantial number of electrodes.

It is also quite conventional for a patient moving through different areas of a hospital to have to be connected to equipment at these areas and, on some occasions, the equipment is moved with the patient on a trolley or the like, bearing in mind that there is physical connection between the patient and the equipment and, on other occasions, the electrodes are disconnected from the equipment, the patient is moved to another place where connections are remade to the electrodes.

Both of these arrangements are unsatisfactory.

To move the equipment with the patient means that the equipment is moved from its normal position and it can, on occasions, be quite difficult to recover pieces of equipment which have been moved to different places in the hospital when they are required for use at their home position.

The second situation is not greatly satisfactory as there is intrusion upon the patient as the electrodes are connected and disconnected and, whilst this happens, there is also the possibility that the connection between the patient and the electrode can be disturbed.

The object of the invention is to provide means whereby a patient's status can readily be monitored but which only necessitates an initial connection of electrodes and other devices for the patient.

The invention includes, in its broadest sense, an electrode assembly for connection to a patient's body and a transmitter for operable connection to the electrode assembly to receive the output therefrom and transmit these to an adjacent receiver associated with monitoring equipment.

In a particular form of the invention, the electrodes of the assembly are associated with a surface which is adhesive covered and adapted for connection to the patient's body but which, prior to use, is provided with a removeable backing, the terminations of the electrodes being adapted to be connected to the transmitter which can be brought into contact with the electrode assembly and retained in contact therewith. an electrode assembly adapted to be connected to a patient's body and a transmitter which can be connected to the electrode assembly to obtain the results of the electrode's output and transmit these to a closely adjacent position for use in monitoring equipment.

In one form the electrode assembly is provided with an adhesive surface which is normally covered by a removeable backing and which has the electrodes embedded therein or located thereon, the terminations of the electrodes being adapted to be connected to a transmitter which can be brought into contact with the electrode assembly and retained therewith.

A second aspect of the invention is the provision of a method of monitoring vital signs of a patient including placing at least one electrode assembly on the patient's body, electrically connecting to the electrode assembly a device to monitor and convert the signal from the electrodes to a required form for transmission, transmitting the converted signal, receiving the transmitted signal and using this to operate equipment designed to monitor the function for which the electrodes are specific.

In this specification, we will refer specifically to electrodes and electrode assemblies, but it is to be appreciated that the invention is equally applicable to any form of transponder which obtains a signal from the skin, or even from beneath the skin, such as the pulse or certain blood monitoring functions and for the purpose of this specification, all transponders are deemed to be incorporated in the expression electrodes.

In order that the invention may be more readily understood we shall describe one embodiment of this in relation to the accompanying drawings, in which:

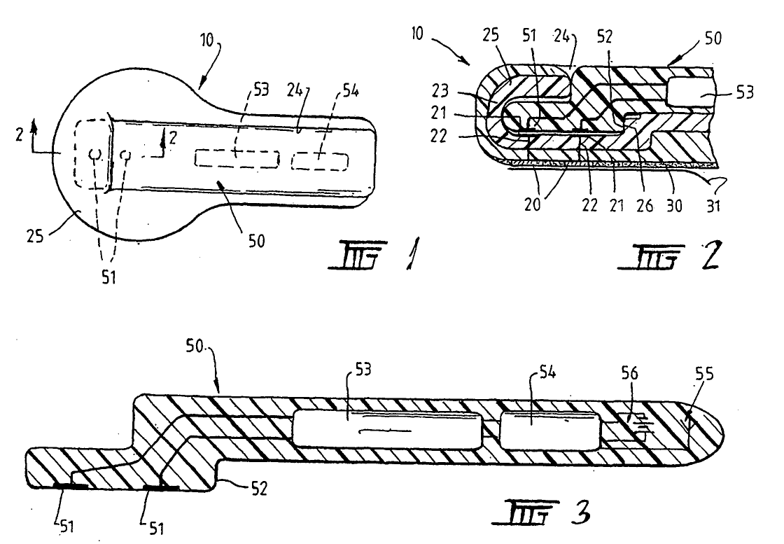

FIG. 1 is an indicative top view of the electrode assembly with the transmitter extending outwardly therefrom;

FIG. 2 is a section along line 2-2 of FIG. 1 showing the arrangement of the electrode assembly and the inter-engagement of this with the transmitter; and

FIG. 2 is a schematic view of the transmitter separate from the electrode assembly.

The system comprises two components. The electrode assembly 10 which is a throwaway component and which has at least one electrode 20 located in what will be the underside thereof when connected to a patient, and which have contacts 21 which are in connection with the electrodes by way of conductors 22. These contacts 21 extend into the body and are part of a connector 23 which is formed of a plastics material or the like.

In the particular application of the invention, the connector is in a sleeve 24 or slot formed in the material 25 of the electrode as will be described further hereafter.

As is conventional in the medical electronics art, the electrode assembly is provided with an adhesive 30 on the face which is to be in contact with the patient which adhesive, in turn, has a protective backing or release sheet 31 thereon which is removed before connection. The electrodes 20 preferably extend slightly beyond the adhesive 30 so as to make good contact with the patient.

Normally such electrode assemblies are coated with or attached to a portion of the patient's body which is being monitored with a conducting material and the adhesive has to be sufficiently aggressive to permit connection notwithstanding such material.

The transmitter portion has a body 50 which may be of a plastics material which pots the electronic components, and also acts as a connector. Shown very schematically in FIG. 3, the body has contacts 51 which are adapted to connect with the contacts 21 associated with the electrodes. As shown, the contacts are surfaces which are in close relationship with each other but they may preferably be pin and socket connectors. The component 53 may have a circuit board to receive the signals from the electrodes 21 and means whereby the signals can be prepared for transmission. There is also a transmitter 54 having an antenna 55, which can be a very short range transmitter, which transmits the prepared signal over approximately 1 metre or less. There is also a battery power supply 56 which powers both components which, in practice can be on a single board.

This transmitter assembly is contacted with the electrode assembly by passing it into the sleeve 24 ant the connecters move together until the shoulder 52 on the transmitter passes over the shoulder 26 of the electrode assembly and the contacts make. Because of the shoulders 26 and 52, once the assembly is completed, the two components are retained together. The arrangement of connection is such that the transmitter is not readily displaced from the electrode assembly.

In operation, the extended portion of the transmitter is passed through the sleeve or slot of the electrode and moved into position where it is effectively locked at which position the contacts of the electrode and those of the device make and the signal received by the electrode is thus transmitted to the printed circuit board of the device where it is treated appropriately and forwarded to the transmitter for reception by a spaced receiver. Depending on requirements, part of the transmitter assembly can pass beyond the body of the electrode assembly if this is required to ensure good transmission of the signal generated by the electrodes.

The transmitter and its associated circuitry may provide either an analogue or a digital signal based on the intensity of the signal from the electrodes and this signal is transmitted to a closely adjacent receiver which is provided with decoding means which enables the signal to be reconstituted and its value ascertained and, if required, continuously monitored.

The transmitter has a very short range, say of the order of 1 metre, but this is sufficient for the signal to be received by a bedside decoder which incorporates a receiver for the signal and a decoder to turn it into a signal of the type provided by the electrodes before modification, or can be adapted to consider the modified form directly. That is, the output from the decoder device can be used as the input for an apparatus which would normally receive signals from the type of electrode assemblies.

Whilst we have referred, generally, to an electrode to be connected to a patient's body, the use of the electrodes can be varied.

For example, they could be for use in an ECG machine, could meter body temperature, respiration (rate and rhythm), saturated oxygen, saturated carbon dioxide and sodium potassium and calcium measurements. These latter are rather esoteric and new but others, such as ECG and heart and respiratory rate, are well known in the medical art.

Because of the arrangement it would be quite feasible if, say, a patient had a heart attack or stroke, for ambulance officers in a MICA ambulance could attach electrodes to the patient when he or she is picked up and could be given a continuous reading of the vital signs for which the electrodes are adapted whilst the patient is in an ambulance. These may, if required be passed directly to the receiving hospital, When the ambulance reaches the hospital it is only necessary for the patient to be removed and brought into the emergency department at which time measurements can be continued by locating a satisfactory decoder close to the patient. The patient can be moved to other areas, such as X-ray or intensive care and have a required decoder located close by them so that the measurements can be maintained effectively, continuously.

The use of very low power transmitters in association with electrodes means that there is little interference with any other equipment in the hospital so that there is no restriction on a number of patients which can have electrodes of the general type connected to them. Further, the electrodes can, if required, each be individually coded so their signals are received by only the decoder adapted to receive the particular coding and thus there is no possibility of confusion as to the patient from whom the signals emanated.

I have described one particular form of electrode and transmitter unit but it is to be understood that this are purely exemplary. It is desirable that the electrode assembly and transmitter unit can be readily connected one to the other so that the signal from the electrode assembly is reliably passed to the transmitter but they can readily be separated when the electrodes are removed from the patient and the electrodes assemblies are to be disposed of.

Also, the transmitter unit can be fully sealed so that it can be sterilised after use and it may be that the unit is such that when the battery ceases to operate, the unit it disposable.

I claim:

1. An electrode assembly for connection to a patient's body and a transmitter for operable correction to the electrode assembly to receive the output therefrom and transmit these to an adjacent receiver associated with monitoring equipment.

2. An assembly as claimed in claim 1 wherein the electrodes of the assembly are associated with a surface which is adhesive covered and adapted for connection to the patient's body but which, prior to use, is provided with a removeable backing, the terminations of the electrodes being adapted to be connected to the transmitter which can be brought into contact with the electrode assembly and to retained in contact therewith.

3. An assembly as claimed in claim 1 or claim 2 wherein there is a contact member at which the electrodes terminate which contact member can engage with a complementary contact member on the transmitter.

4. An assembly as claimed in claim 3 wherein the engagement of the contact members holds the transmitter relative to the electrode assembly.

5. An assembly as claimed in claim 3 or claim 4 wherein one of the contact members includes a socket having connectors therein and the other contact member has a member, complementary in shape to the socket the engage therewith.

6. An assembly as claimed in any preceding claim 3 to 5 wherein the electrode assembly includes a sleeve which can receive and retain the transmitter, the contact members being in electrical engagement when the transmitter is fully located in the sleeve.

7. An assembly as claimed in any preceding claim wherein the electrode assembly is a one-use device.

8. An assembly as claimed in claim 7 wherein the transmitter can be re-used.

9. An assembly as claimed in claim 9 wherein the transmitter can be sterilized after use.

10. A method of monitoring vital signs of a patient including placing at least one electrode assembly on the patient's body, electrically connecting to the electrode assembly a device to monitor and convert the signal from the electrodes to a required form for transmission, transmitting the converted signal, receiving the transmitted signal and using this to operate equipment designed to monitor the function for which the electrodes are specific.

11. A method as claimed in claim 10 wherein the monitoring equipment can directly read the signal transmitted as the function transmitted.

12. A method as claimed in claim 10 wherein the transmitted signal is re-converted to the form of a signal provided by the electrodes and this signal is monitored by equipment designed to monitor the appropriate function.
